(19) United States
(12) Patent Application Publication
(10) Pub. No.: US 2011/0253130 A1
(43) Pub. Date: Oct. 20, 2011

(54) Bikers lap belt

(76) Inventor: Darrell Dean Yarnall, Garden City, KS (US)

(21) Appl. No.: 12/799,219

(22) Filed: Apr. 20, 2010

(51) Int. Cl. A61F 5/28

(52) U.S. Cl. 128/99.1

(57) ABSTRACT

The Bikers Lap Belt, hereafter known as the belt, was road tested for two (2) years. It was found that when wearing the belt on long motorcycle trips (200 miles or more), the experienced motorcycle rider of about fifty four (54) years of age, found the following to be true: The rider had less muscle fatigue in the upper and lower back, no pain in the hips, and no fatigue in the legs. The motorcycle was easier to control in windy conditions at high speeds, especially when passing large trucks requiring less muscle movement of the legs. Muscle tension was less in all conditions. When riding without the belt, the rider experienced upper back pain and fatigue, substantial hip pain at the end of several hours of riding, and severe overall body fatigue. The belt gives riders the ability to ride longer with more comfort and less joint or muscle pain.

### BACKGROUND OF THE INVENTION

Help lessen back, hip, and knee pain for the comfort of motorcycle riders while engaged in riding on a motorcycle. Further, increase endurance and lesson fatigue when riding on a motorcycle.

### SUMMARY OF THE INVENTION

Currently, motorcycle riders and their passengers endure discomfort after riding for long periods of time. Riders become fatigued because they are sitting or straddling a motorized two wheeled vehicle exposing themselves to outside natural elements such as wind resistance and gravity while moving in a forward motion on a street or highway. While operating or riding a motorcycle, the rider uses various muscle groups within their body to stay on the motorcycle and remain balanced while it is in motion. In an effort to remain upright and balanced, the rider's muscles become fatigued and stressed, especially in the back, hip, and knee regions of the human body.

The Bikers Lap Belt, is worn around the upper thighs and below the hips of the riders like a person wears a belt to keep his pants up. The Bikers Lap Belt holds and supports their legs against the forces of wind and gravity pressure when riding a motorcycle in a forward direction. The rider is able to relax the main muscle groups of the human body by resting the upper legs against the belt's tension regardless of the velocity of the wind against his body. The Bikers Lap Belt is not worn around the waist, but is placed around a line immediately under the cheeks of the human buttocks and fastened in front of the groin or crotch of the human body. The rider or passenger can ride great distances with less stress and muscle fatigue. Road testing this Bikers Lap Belt has shown to lesson muscle pain in the back, hips, and knees or eliminated muscle discomfort and/or fatigue all together. The Bikers Lap Belt does not interfere with the operation the motorcycle and is designed with a quick disconnect for safety.

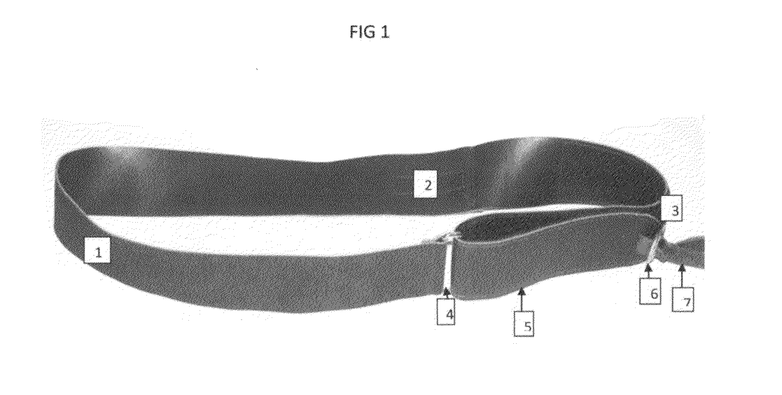

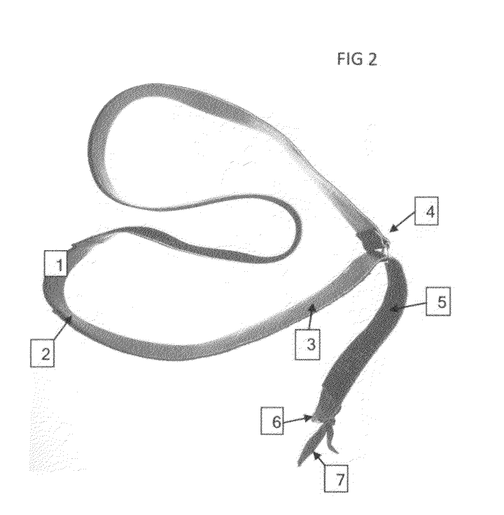

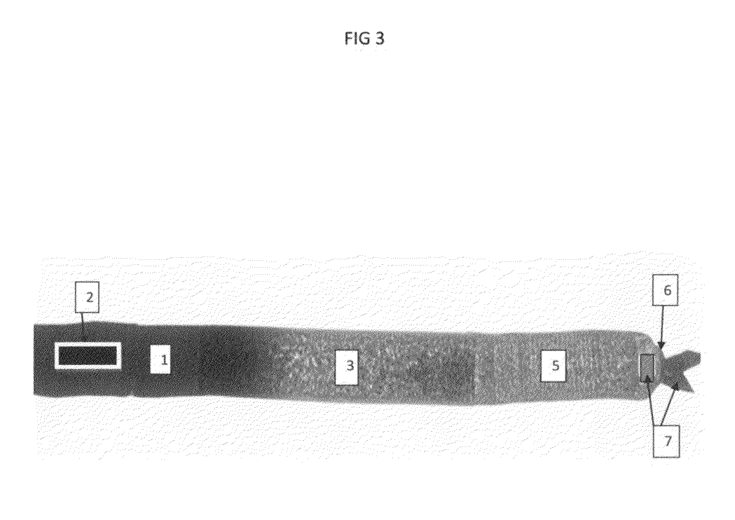

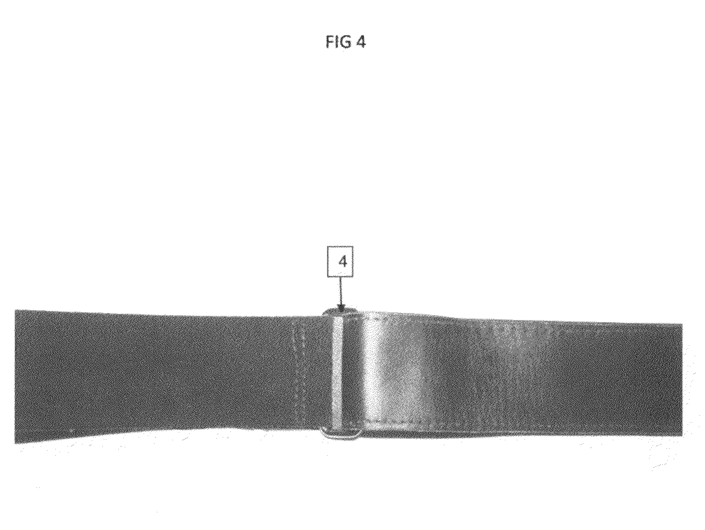

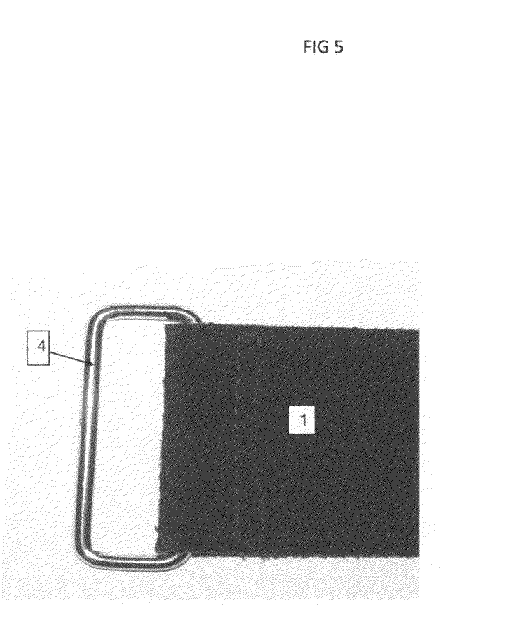

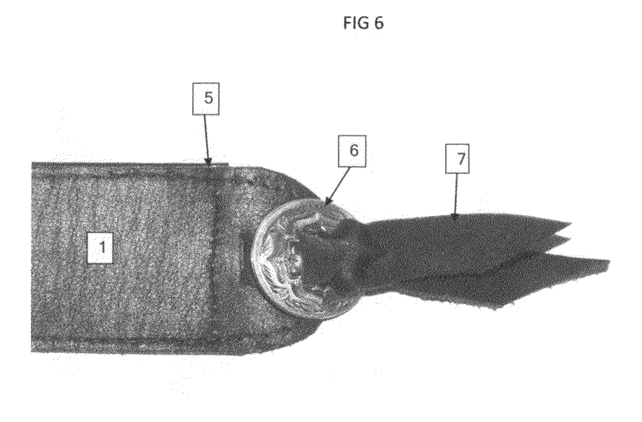

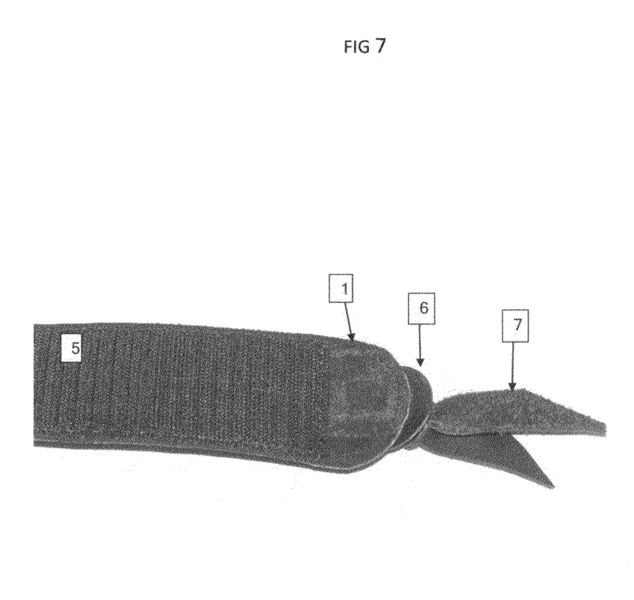

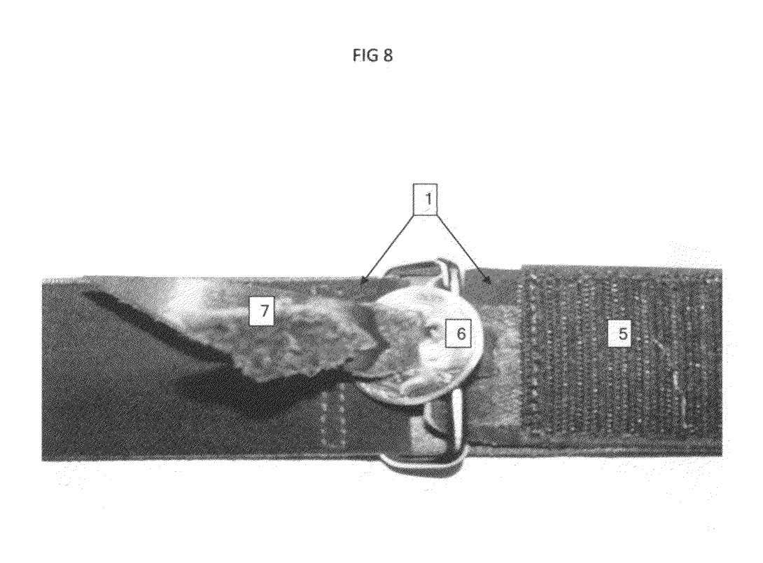

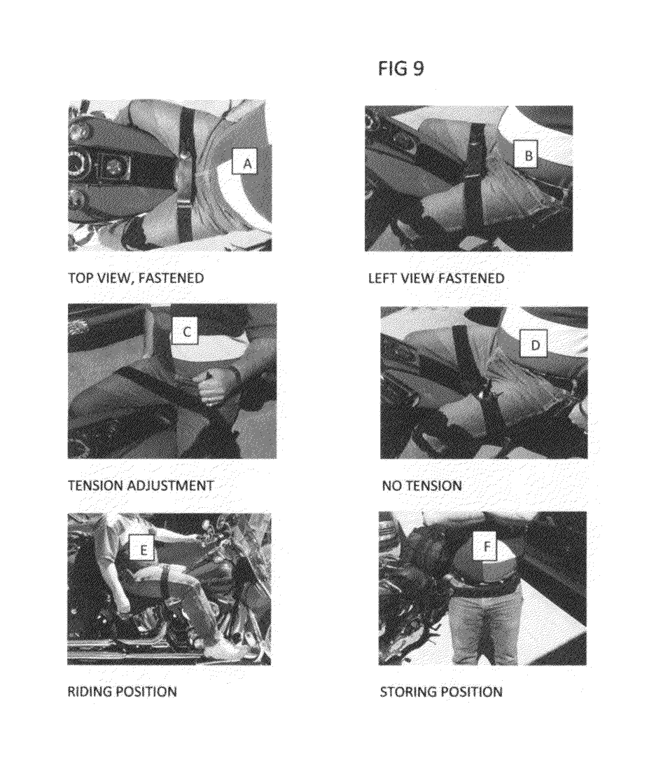

### DESCRIPTION OF DRAWINGS

| REFERENCE NUMBER | NAME OF PART |
|---|---|
| FIG. 1 | Front view, closed, Bikers Lap Belt |
| 1 | Main Body Leather Strap |
| 2 | Back stitching of loop material |
| 3 | Loop material |
| 4 | Rectangle D Ring |
| 5 | Back stitching of hook material |
| | Slotted round metal Concho |
| 7 | Leather thong |
| FIG. 2 | Top view, open, Bikers Lap Belt |
| 1 | Main Body Leather Strap |
| 2 | Back stitching of loop material |
| 3 | Loop material |
| 4 | Rectangle D Ring |
| 5 | Back stitching of hook material |
| | Slotted round metal Concho |
| 7 | Leather thong |
| FIG. 3 | Partial back/inside view, fastening end of belt |
| 1 | Main Body Leather Strap |
| 2 | Back stitching of loop material |
| 3 | Loop material |
| 5 | Hook material |
| 6 | Concho |
| 7 | Leather thong |
| FIG. 4 | D ring assembly in the fastened and/or |
| | closed mode |
| 1 | Main body leather strap |
| 4 | D ring |
| FIG 5 | Frontal view, D ring attachment, |
| | opposite end of fastened end. |
| 1 | Main body leather strap |
| 4 | D ring attached |
| FIG. 6 | Frontal view of thong and Concho |
| 1 | Main body leather strap, fastening end |
| 5 | Hook material, other side |
| 6 | Concho |
| 7 | Thong |
| FIG. 7 | Inside view fastening end |
| 1 | Main body leather strap |
| 5 | Hook material |
| 6 | Concho |
| 7 | Thong |
| FIG. 8 | Frontal view of open belt stop mode |
| 1 | Main body leather strap |
| 5 | Hook material |
| 6 | Concho |
| 7 | Thong |
| FIG. 9 | Color Photos A, B, C, D, E, F |
| A | Top View Fastened |
| B | Left View Fastened |
| C | Tension Adjustment |
| D | No Tension |
| E | Riding Position |
| F | Storing/Carrying Position |

### DETAILED DESCRIPTION OF THE INVENTION

The Bikers Lap Belt, hereafter referred to as the “belt”, is configured and designed for a human person, hereafter referred to as a “rider”, to wear upon his person during the riding or driving of a two wheeled vehicle, commonly known as a motorcycle, hereafter referred to as a “cycle”, while the cycle is in motion on a street or highway moving in the forward direction at any speed.

The belt is designed to provide support to the upper legs by supporting the upper thigh region of the rider and holding the legs in any position the rider desires for comfort. Because of this support, the belt provides the rider a time to relax his back and leg muscles, therefore eliminating fatigue normally caused by riding a cycle.

The belt is worn like other belts used in securing trousers to the waist of any rider with the following exception: The belt is worn by stepping into the belt, pulling the belt up to upper thigh area of the rider's body and fastening the belt around the rider's upper thighs positioned just below the buttocks cheeks in the back of the rider and across the groin and/or crotch in front of the rider. The belt is not fastened at this point and loose enough for the rider to mount the cycle and get seated. Once seated, the belt can be adjusted to a comfortable tension once the cycle is in motion. The belt's tension can be changed at any time during the riding activity. To tighten or change the belt's tension, pull on the Concho/thong and separate the hook and hoop closure and pull on the belt to tighten and push the belt the opposite way through the D ring to loosen the belt. Reattached the hook and loop closure. This process is aided by pushing against the belt in an outward direction with your legs or pulling your legs together while pulling on the Concho end of the belt.

Although the methods of assembly of this belt can vary, the following description was found to be the best assembly for this invention. The belt's components/parts, consist of the following:

(SEE FIG. 1.) Part number 1 is a 4 oz in weight (leather manufacturing measure), leather strap made from finished cowhide leather about two (2) inches wide and sixty (60) inches in length. One side is rough in texture and the other side is smooth in texture. It should be noted that Part #1 can be made out of any animal or reptile skin, leather, synthetic, cloth, or pliable material, and any textile or manmade material of any length, thickness, or width, and have smooth or rough side finishes.

(SEE FIGS. 1 and 3) Part number 2, is a strip of loop material (Velcro) about three (3) inches in length and three quarter (¾) inches in width and sewn on the rough side of Part number 1 about twenty six (26) inches from the Concho end of the belt. This part serves to fasten the belt around the waist of the person for carrying the belt when not riding on the cycle. This material can be placed at any length on Part number 1 to fit different size riders.

(SEE FIGS. 1, 2 and 3) Part number 3 is loop material (Velcro) sewn on to the rough side of Part number 1. It is about two (2) inches wide and fifteen (15) inches in length. It is placed on the rough side of the belt beginning at about nine (9) inches from the Concho end of the belt.

(SEE FIGS. 1, 2, 4, 5 and 8) Part number 4 is a wire ring and/or D ring measuring about two (2) inches in length and one half (½) inch opening shaped like a rectangle. The D ring is about one eighth (⅛) inch thick. This D ring allows adjustment of the belt when riding the cycle. Part number 1 slides through this D ring. The D ring can be made of plastic or other man made or textile materials. The D ring is attached to Part number 1 at the opposite end of the Conch end of the belt. It is sewn on by looping the end of the belt through the D ring and sewing the end of the belt to the smooth side of Part number 1.

(SEE FIGS. 1, 2, 3, 6, 7 and 8) Part number 5 is a loop material (Velcro) about two (2) inches wide and eight (8) inches in length. It is sewn on Part number 1 on the rough side beginning at the Concho end of the belt. It is placed about one (1) inch from the end of the belt and sewn.

(SEE FIGS. 1, 2, 3, 6, 7 and 8) Part number 6 is a slotted Concho made out of metal material and used as jewelry, decoration, or for fastening lanyards and straps to other leather products. It is commonly known as an instrument or fastening device as noted in Western Folklore. This Concho has two (2) slots cut in the center for tying to a thong or strap and is concaved. The Concho serves as a stop which does not allow the end of the belt to slip through the D ring (Part number 4) if released by the rider during forward motion while riding the cycle preventing the ends of the belt to swing freely in the wind. It also serves as an obvious point of release if the rider wants to release the tension of the belt. The rider simply pulls the Concho upward to release or unfasten the hook and loop material. Part number 6, can be any instrument resembling a Concho or any object that cannot slip through the D ring when the tension is released. It can be made out of any material, manmade or natural, or any textile that can be tied to a thong or lanyard.

(SEE FIGS. 1, 2, 3, 6, 7 AND 8) Part number 7 is a leather thong (lanyard) and attached to Part number 1 at the Concho end of the belt and attaches Part number 6, the Concho. The thong measures about one half (½) inch in width and about seven (7) inches long and is about four (4) OZ. weight by leather standards of measure. It has one (1) rough side and one (1) finished side. It is cut from the same leather batch Part number 1 is made from. To attach the thong, two slots are cut on the Concho end of Part number 1 in a horizontal position with Part number 1's and parallel to its' length. The slots are about one half (½) inches long and about one half (½) inch apart and beside each other in parallel. The thong is tapered on the ends. The ends are inserted from the direction of the rough side of Part number 1. One (1) end of the thong is inserted in each slot on Part number 1. After the thong ends are inserted through Part number 1 and the Conchos's slots, they are tied in a braided fashion until secure. The thong is now attached to Part number 1, the Concho, and tied within its self holding the assembly together.

The belt can be decorated to the individual rider's liking. It can be made of any color. The texture of both sides of the belt can be rough or smooth.

(SEE FIG. 9) Photo A shows the proper riding position with the belt fastened. Photo B shows the left side view when wearing the belt in the proper position and tension is applied and fastened. Photo C shows the left hand of the rider pulling up and applying tension to the belt holding the Concho end of the belt. Photo D shows the belt with no tension applied. Photo E shows a side view of rider on the motorcycle wearing the belt in the proper position. Photo F shows the rider dismounted from the motorcycle and wearing the belt in a carry position and not engaged.

1. The Bikers Lap Belt allows the rider to relax some major muscle groups in the human body when riding a motorcycle; therefore the rider enjoys more comfort and less fatigue while engaged in extended travel.
